(19) United States
(12) Patent Application Publication
(10) Pub. No.: US 2024/0370607 A1
(43) Pub. Date: Nov. 7, 2024

(54) METHOD AND SYSTEM FOR PREDICTING GRAIN REFINEMENT OF MACHINED SURFACE OF TITANIUM ALLOY SUBJECTED TO ULTRA-PRECISION CUTTING

(71) Applicant: Guangdong University of Technology, Guangzhou City (CN)

(72) Inventors: Zhanwen SUN, Guangzhou City (CN); Jie JIAO, Guangzhou City (CN); Sujuan WANG, Guangzhou City (CN); Haiqing WU, Guangzhou City (CN)

(21) Appl. No.: 18/655,688

(22) Filed: May 6, 2024

(30) Foreign Application Priority Data
May 6, 2023 (CN) 202310504919.3

(51) Int. Cl. G06F 30/20

(52) U.S. Cl. CPC G06F 30/20

(57) ABSTRACT

Provided is a method and system for predicting grain refinement of a machined surface of titanium alloy subjected to ultra-precision cutting. The method includes: obtaining an α-phase crystal parameter of a titanium alloy workpiece to be machined in advance, and selecting a grain refinement analysis region on the titanium alloy workpiece to be machined; establishing a discrete dislocation dynamics model corresponding to the grain refinement analysis region according to the α-phase crystal parameter, where the discrete dislocation dynamics model is configured to simulate dislocation behavior in the grain refinement analysis region; and performing grain refinement prediction analysis on the grain refinement analysis region according to the discrete dislocation dynamics model in response to ultra-precision cutting of the titanium alloy workpiece to be machined, and obtaining a corresponding grain size after cutting.

### CROSS REFERENCE TO RELATED APPLICATION

This patent application claims the benefit and priority of Chinese Patent Application No. 202310504919.3, filed with the China National Intellectual Property Administration on May 6, 2023, the present disclosure of which is incorporated by reference herein in its entirety as part of the present application.

### TECHNICAL FIELD

The present disclosure relates to the technical field of titanium alloy cutting, and particularly relates to a method and system for predicting grain refinement of a machined surface of titanium alloy subjected to ultra-precision cutting.

### BACKGROUND

In a cutting process of titanium alloy, the interaction between a tool and a workpiece can produce a grain refinement effect on a machined surface, which affects the mechanical properties of the machined surface, and then directly affects the design of the subsequent machining process.

A current method for predicting and evaluating grain refinement in titanium alloy machining is usually an experimental testing method for observation and evaluation, which is not only complicated, but also takes a long time and high cost. Although a cutting simulation model based on molecular dynamics and a finite element method can dynamically simulate an ultra-precision cutting process, this model cannot predict the crystal structure evolution during cutting due to the limitation to the simulation scale and simulation principle, which makes it difficult to intuitively and quickly evaluate the quality of a machined surface and cannot guide the design of the subsequent machining process.

Therefore, it is urgent to provide an efficient and accurate method for predicting the degree of grain refinement of a machined surface of titanium alloy during ultra-precision cutting.

### SUMMARY

An objective of the present disclosure is to provide a method for predicting grain refinement of a machined surface of titanium alloy subjected to ultra-precision cutting. Dislocation behavior inside titanium alloy during machining is calculated and simulated through discrete dislocation dynamics, a microstructure change of titanium alloy grain refinement is deduced, and a grain size of the machined surface is predicted, such that application defects of a prior method for predicting grain refinement of machined surface of titanium alloy subjected to cutting are overcome, the quality of the machined surface can be intuitively, rapidly and accurately evaluated, which provides reliable guarantee for design guidance of a subsequent machining process and has high application value.

In order to achieve the above objectives, it is necessary to provide a method and system for predicting grain refinement of a machined surface of titanium alloy subjected to ultra-precision cutting, to address the above technical problems.

In a first aspect, an embodiment of the present disclosure provides a method for predicting grain refinement of a machined surface of titanium alloy subjected to ultra-precision. The method includes:
obtaining an α-phase crystal parameter of a titanium alloy workpiece to be machined in advance, and selecting a grain refinement analysis region on the titanium alloy workpiece to be machined;
establishing a discrete dislocation dynamics model corresponding to the grain refinement analysis region according to the α-phase crystal parameter, where the discrete dislocation dynamics model is configured to simulate dislocation behavior in the grain refinement analysis region; and
performing grain refinement prediction analysis on the grain refinement analysis region according to the discrete dislocation dynamics model in response to ultra-precision cutting of the titanium alloy workpiece to be machined, and obtaining a corresponding grain size after cutting.

Further, the step of selecting a grain refinement analysis region on the titanium alloy workpiece to be machined includes:
selecting the grain refinement analysis region with a preset size on the titanium alloy workpiece to be machined according to an initial cutting position of a cutting tool, where the grain refinement analysis region is located just below the initial cutting position.

Further, the α-phase crystal parameters include dislocation source density, a spacing between a dislocation source and an obstacle, and a glide plane spacing; and
the step of establishing a discrete dislocation dynamics model corresponding to the grain refinement analysis region according to the α-phase crystal parameter includes:
establishing an α-phase crystal glide system according to a glide plane spacing and a glide direction in an α phase of titanium alloy, and a relative angle between a glide system and a grain boundary in a unit cell, where glide system directions of the α-phase crystal glide system include a 0° direction, a 60° direction and a −60° direction;
dividing the grain refinement analysis region into a preset number of grain regions with a same size evenly;
obtaining a number of dislocation sources according to the dislocation source density and a size of the grain refinement analysis region, distributing the dislocation sources in the glide system directions through a normal distribution method evenly, and obtaining positions of all the dislocation sources;
arranging one dislocation obstacle in front of and behind each dislocation source in the glide system direction according to the spacing between a dislocation source and an obstacle separately, and obtaining positions of all the dislocation obstacles; and
establishing the discrete dislocation dynamics model based on discrete dislocation dynamics according to the α-phase crystal glide system, the positions of all the dislocation sources, and the positions of all the dislocation obstacles.

Further, the steps of performing grain refinement prediction analysis on the grain refinement analysis region according to the discrete dislocation dynamics model, and obtaining a corresponding grain size after cutting include:
obtaining ultra-precision cutting parameters, where the ultra-precision cutting parameters include a corner radius, a cutting speed and a cutting depth;
obtaining a calculation prediction time according to the cutting speed and a length of the grain refinement analysis region, taking each dislocation source as an immovable dislocation, and initializing a number of dislocations, where the dislocations include movable dislocations and immovable dislocations; and
performing iterative analysis on grain refinement in the grain refinement analysis region based on the discrete dislocation dynamics model within the calculation prediction time according to the ultra-precision cutting parameters and the α-phase crystal parameters when a cutting tool cuts to the grain refinement analysis region, and obtaining the grain size after cutting.

Further, the α-phase crystal parameters further include a shear modulus, a Poisson ratio of titanium alloy, a Burgers vector, a dislocation segment length, a dislocation multiplication time, a viscosity coefficient and dislocation obstacle strength; and
the steps of performing iterative analysis on grain refinement in the grain refinement analysis region based on the discrete dislocation dynamics model within the calculation prediction time, and obtaining the grain size after cutting include:
initializing a number of immovable dislocations, a number of movable dislocations and a number of times of grain refinement;
obtaining cutting thrust of the cutting tool at a current moment, and obtaining a shear stress and a long-range action resultant stress of each dislocation and each dislocation source according to the cutting thrust and the position of each dislocation;
calculating an intra-region Peierls-Nabarro stress and a dislocation source multiplication intensity in the grain refinement analysis region according to the shear modulus, the Poisson ratio of titanium alloy, the Burgers vector and the dislocation segment length, where the intra-region Peierls-Nabarro stress is expressed as:

$${σ}_{P - N} = \frac{2 μ}{1 - v} {e}^{\frac{- 4 π ξ}{b}}$$

where σ_{P-N} denotes the intra-region Peierls-Nabarro stress, and μ, v, b and ξ denote the shear modulus, the Poisson ratio of titanium alloy, the Burgers vector and a dislocation half-width respectively; and
the dislocation source multiplication intensity is expressed as:

$${τ}_{s} = \frac{μ b}{{L}_{ab}}$$

where τₛ denotes the dislocation source multiplication intensity, and L_{ab} denotes the dislocation segment length;
obtaining a corresponding dislocation source resultant stress according to the shear stress and the long-range action resultant stress of each dislocation source, where the dislocation source resultant stress is expressed as:

$${σ}_{source}^{k} = {τ}_{ok} + {σ}_{ok} , k = 1 , … , {n}_{s}$$

where τₒₖ, τₒₖ and σ_{source}^{k} denote a shear stress, a long-range action resultant stress and a dislocation source resultant stress of a kth dislocation source respectively;
determining whether the dislocation source resultant stress is great than the dislocation source multiplication intensity, if so, keeping a position of the corresponding dislocation source unchanged, generating a pair of positive and negative dislocations on the glide system of the corresponding dislocation source according to a preset distance, and increasing the number of movable dislocations;
obtaining a movement speed of each movable dislocation according to the shear stress, the long-range action resultant stress and the intra-region Peierls-Nabarro stress of each movable dislocation, obtaining corresponding obstacle strength according to the movement speed of each movable dislocation and positions of dislocation obstacles of the same glide system, and obtaining a current position of the corresponding movable dislocation according to the movement speed and the obstacle strength;
obtaining a corresponding movable dislocation spacing according to the current position of each movable dislocation, determining whether dislocation annihilation occurs according to the movable dislocation spacing, removing the corresponding movable dislocation when the dislocation annihilation occurs, and reducing the number of movable dislocation;
counting a number of grain boundary dislocations in each grain region according to the current positions of all movable dislocations and the positions of the dislocation sources, and obtaining a corresponding grain boundary torsion angle according to the number of grain boundary dislocations in each grain region;
determining whether a grain refinement region exists according to the grain boundary torsion angle, dividing the grain refinement region into four grain sub-regions with a same size, and increasing the number of times of grain refinement; and
updating a current analysis moment according to the dislocation multiplication time, obtaining current cutting thrust of the cutting tool, updating the cutting thrust according to the current cutting thrust, continuing a next round of grain refinement analysis, stopping iteration until a preset calculation prediction time is reached, and obtaining the grain size after cutting according to the number of times of grain refinement.

Further, the step of obtaining a shear stress and a long-range action resultant stress of each dislocation and each dislocation source according to the cutting thrust and the position of each dislocation includes:
obtaining the shear stress of each dislocation according to the cutting thrust, where the shear stress is expressed as:

$${τ}_{i} = {σ}_{t} - {𝓏e}^{λ \sqrt{{[ {x}_{i} - ( u - {v}_{t} {t}_{0} ) ]}^{2} + {( {y}_{i} - w )}^{2}}}$$

in the equation,

$${σ}_{t} = \frac{{F}_{t}}{W} \\ W = {R}^{2} \cdot arccos \frac{R - h}{R} - ( R - h ) \sqrt{{R}^{2} - {( R - h )}^{2}}$$

where τᵢ denotes a shear stress caused by cutting on an ith dislocation; z and λ denote material parameters of titanium alloy; νₜ denotes the cutting speed; (xᵢ, yᵢ) denotes coordinates of the ith dislocation in the grain refinement analysis region; u and w denote a length and a width of the grain refinement analysis region respectively; Fₜ denotes the cutting thrust; σₜ denotes the shear stress of the cutting tool; S denotes a contact area between the cutting tool and the titanium alloy workpiece to be machined; R denotes the corner radius; and h denotes the cutting depth; and
obtaining a long-range action force between dislocations according to the position of each dislocation, and obtaining the long-range action resultant stress of each dislocation according to the long-range action force between dislocations, where the long-range action resultant stress is expressed as:

$${σ}_{all}^{i} = {\sum }_{j = 1}^{i \neq j} {σ}_{ij}$$

in the equation,

$${σ}_{ij} = \frac{μ b}{2 π ( 1 - v )} \cdot \frac{- 2 {dy}_{ij} \cdot sin 2 θ \cdot {dx}_{ij}^{2} + {dx}_{ij} \cdot cos 2 θ \cdot ( {dx}_{ij}^{2} - {dy}_{ij}^{2} )}{( {dx}_{ij}^{2} + {dy}_{ij}^{2} )} , i , j = 1 , … , {n}_{s} + {n}_{dis} , i \neq j$$

where σₐₗₗⁱ denotes a long-range action resultant stress of the ith dislocation; σᵢⱼ and θ denote a long-range action force and an angle between the ith dislocation and a jth dislocation respectively; dxᵢⱼ and dyᵢⱼ denote distances between the dislocation i and the dislocation j along an x axis and an y axis respectively; and nₛ and n_{dis} denote the number of immovable dislocations and the number of movable dislocations respectively.

Further, the step of obtaining a current position of the corresponding movable dislocation according to the movement speed and the obstacle strength includes:
obtaining a first movable dislocation position according to the movement speed and an initial position of each movable dislocation, where the first movable dislocation position is expressed as:

$${s}_{{t}_{0} + t}^{{n}_{l}} = {s}_{{t}_{0}}^{{n}_{l}} + {v}_{{n}_{l}} t , {n}_{l} = 1 , … , {n}_{dis}$$

in the equation,

$${v}_{{n}_{l}} = \frac{( {τ}_{{n}_{l}} - {σ}_{P - N} + {σ}_{{n}_{l}} ) b}{{B}_{g}}$$

where νₙ_{ₗ}, τₙ_{ₗ} and σₙ_{ₗ} denote a movement speed, a shear stress and a long-range action resultant stress of a nₗ th movable dislocation respectively; σ_{P-N} denotes the intra-region Peierls-Nabarro stress; b and B_{g} denote the Burgers vector and the viscosity coefficient respectively; sₜ_{₀}ⁿ^{ₗ} and sₜ_{₀}_{^{+t}}ⁿ^{ₗ} denote positions of the nₗ th dislocation before and after moving within a first dislocation multiplication time t from moment t₀ respectively;
obtaining a corresponding driving force according to the shear stress and the long-range action resultant stress of each movable dislocation, and obtaining a corresponding movement resistance according to the intra-region Peierls-Nabarro stress and the corresponding obstacle strength; and
determining whether the driving force of each movable dislocation is greater than the corresponding movement resistance, if so, taking the first movable dislocation position as the current position of a movable dislocation, and otherwise, determining that dislocation pile-up occurs in the corresponding movable dislocation, and taking the corresponding initial position as the current position of a movable dislocation.

Further, the grain boundary torsion angle is expressed as:

$${θ}_{b}^{q} = arctan ( b \sqrt{\frac{{n}_{bound}^{q}}{{A}_{bound}^{q}}} )$$

in the equation,

$${A}_{bound}^{q} = ( 1 - \frac{1}{4} ) * {D}^{2}$$

where θ_{b}^{q}, A_{bound}^{q} and n_{bound}^{q} denote a grain boundary torsion angle, a grain boundary region area and a number of grain boundary dislocations of a qth grain region respectively; D denotes a grain size length; and b denotes the Burgers vector.

Further, the grain size after cutting is expressed as:

$${D}_{new} = \sqrt{\frac{u * w}{100 u * 100 w + 3 * n}}$$

where D_{new} denotes the grain size after cutting; n denotes the number of times of grain refinement; and u and w denote a length and a width of the grain refinement analysis region respectively.

In a second aspect, an embodiment of the present disclosure provides a system for predicting grain refinement of a machined surface of titanium alloy subjected to ultra-precision. The system includes:
a preprocessing module configured to obtain an α-phase crystal parameter of a titanium alloy workpiece to be machined in advance, and select a grain refinement analysis region on the titanium alloy workpiece to be machined;
a model establishment module configured to establish a discrete dislocation dynamics model corresponding to the grain refinement analysis region according to the α-phase crystal parameter, where the discrete dislocation dynamics model is configured to simulate dislocation behavior in the grain refinement analysis region; and
an analysis prediction module configured to perform grain refinement prediction analysis on the grain refinement analysis region according to the discrete dislocation dynamics model in response to ultra-precision cutting of the titanium alloy workpiece to be machined, and obtain a corresponding grain size after cutting.

In a third aspect, an embodiment of the present disclosure further provides a computer apparatus, including a memory, a processor, and a computer program stored on the memory and runnable on the processor, where the processor implements steps of the above method when executing the computer program.

In a fourth aspect, an embodiment of the present disclosure further provides a computer-readable storage medium, storing a computer program, where the computer program implements steps of the above method when executed by a processor.

The present application provides a method and system for predicting grain refinement of a machined surface of titanium alloy subjected to ultra-precision cutting. The method achieves a technical solution of obtaining an α-phase crystal parameter of a titanium alloy workpiece to be machined in advance, selecting a grain refinement analysis region on the titanium alloy workpiece to be machined, establishing a discrete dislocation dynamics model corresponding to the grain refinement analysis region according to the α-phase crystal parameter, performing grain refinement prediction analysis on the grain refinement analysis region according to the discrete dislocation dynamics model when the titanium alloy workpiece to be machined is subjected to ultra-precision cutting, and obtaining a corresponding grain size after cutting. Compared with the prior art, in the method for predicting grain refinement of a machined surface of titanium alloy subjected to ultra-precision cutting, the dislocation behavior inside titanium alloy during machining is calculated and simulated based on discrete dislocation dynamics, a microstructure change of titanium alloy grain refinement is deduced, and a grain size of the machined surface after cutting is predicted, such that quality of the machined surface can be evaluated intuitively, rapidly and accurately, reliable guarantee for optimizing a subsequent machining process is provided, and the method has higher application value.

### BRIEF DESCRIPTION OF THE DRAWINGS

FIG. 1 is a schematic flowchart of a method for predicting grain refinement of a machined surface of titanium alloy subjected to ultra-precision cutting according to an embodiment of the present disclosure;

FIG. 2 is a schematic diagram of grain division in a grain refinement analysis region according to an embodiment of the present disclosure;

FIG. 3 is a schematic diagram of intracrystalline dislocation sources fixed on a glide plane according to an embodiment of the present disclosure;

FIG. 4 is a schematic diagram showing that a cutting tool cuts exactly to a selected grain refinement analysis region according to an embodiment of the present disclosure;

FIG. 5 is a detailed flowchart of an iterative calculation process of a grain size after cutting according to an embodiment of the present disclosure;

FIG. 6 is a flowchart for calculating a long-range action resultant stress of a dislocation according to an embodiment of the present disclosure;

FIG. 7 is a schematic diagram of dislocation multiplication according to an embodiment of the present disclosure;

FIG. 8 is a schematic diagram of encounter of a movable dislocation and a dislocation obstacle according to an embodiment of the present disclosure;

FIG. 9 is a schematic diagram of distribution of intracrystalline dislocations after one iteration prediction according to an embodiment of the present disclosure;

FIG. 10 is a schematic diagram of grain refinement after one iteration prediction according to an embodiment of the present disclosure;

FIG. 11 is another schematic flowchart of a method for predicting grain refinement of a machined surface of titanium alloy subjected to ultra-precision cutting according to an embodiment of the present disclosure;

FIG. 12 is a schematic diagram of machined titanium alloy according to an embodiment of the present disclosure;

FIG. 13 is a schematic diagram of dislocation behavior simulation during titanium alloy machining in FIG. 12;

FIG. 14 is a schematic diagram of a grain simulation result of a dislocation behavior simulation result in FIG. 13;

FIG. 15 is a schematic structural diagram of a system for predicting grain refinement of a machined surface of titanium alloy subjected to ultra-precision cutting according to an embodiment of the present disclosure; and

FIG. 16 is an internal structure diagram of a computer apparatus according to an embodiment of the present disclosure.

### DETAILED DESCRIPTION OF THE EMBODIMENTS

In order to make the objectives, technical solutions and beneficial effects of the present application clearer, the present disclosure will be described in further detail below in conjunction with the accompanying drawings and embodiments. Obviously, the embodiments described below are some of the embodiments of the present disclosure and are merely used for illustrating the present disclosure, but are not intended to limit the scope of the present disclosure. Based on the embodiments of the present disclosure, all other embodiments obtained by those of ordinary skill in the art without making creative efforts fall within the scope of protection of the present disclosure.

The method for predicting grain refinement of a machined surface of titanium alloy subjected to ultra-precision provided in the present disclosure is mainly based on discrete dislocation dynamics, real-time simulation and calculation of dislocation behavior of the machined surface in an α-phase of the titanium alloy subjected to ultra-precision cutting are carried out, and grain refinement during machining is deduced, such that a grain size of the machined surface after cutting is accurately predicted, efficient quality evaluation is carried out on the machined surface, and selection of a proper machining process is guided. The following embodiments will describe in detail the method for predicting grain refinement of a machined surface of titanium alloy subjected to ultra-precision cutting.

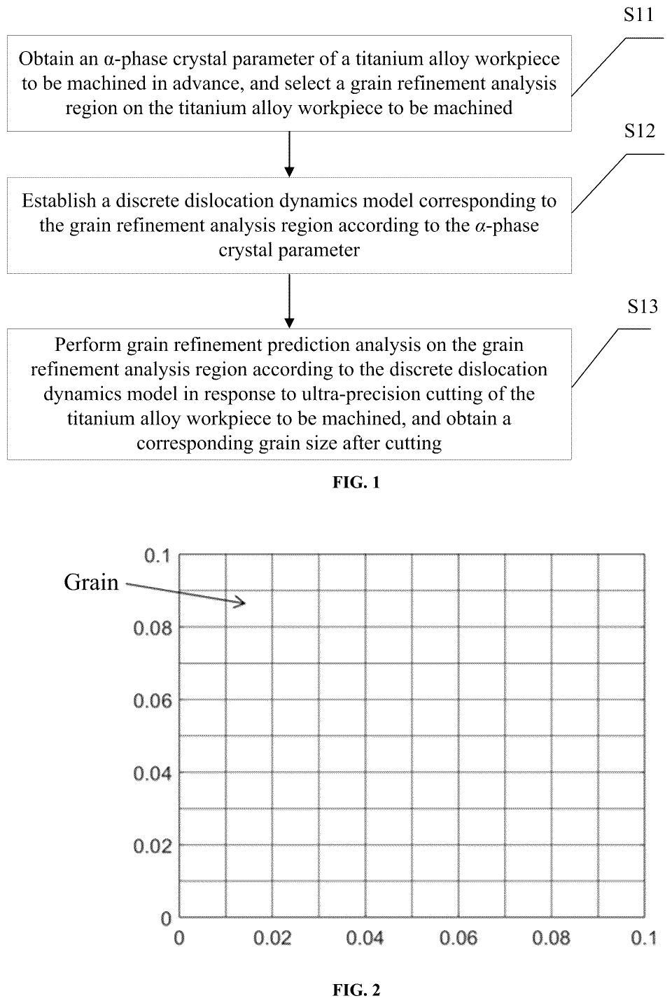

In an embodiment, as shown in FIG. 1, a method for predicting grain refinement of a machined surface of titanium alloy subjected to ultra-precision is provided. The method includes:
S11, obtain an α-phase crystal parameter of a titanium alloy workpiece to be machined in advance, and select a grain refinement analysis region on the titanium alloy workpiece to be machined. The α-phase crystal parameters include dislocation source density, a spacing between a dislocation source and an obstacle, a glide plane spacing, a shear modulus, a Poisson ratio of titanium alloy, a Burgers vector, a dislocation segment length, a dislocation multiplication time, a viscosity coefficient and a dislocation obstacle strength. Correspondingly, an obtaining method can be implemented by using the prior art. For example, the shear modulus (μ) is measured by using a nano-indentation instrument. The Poisson ratio (ν) of titanium alloy is measured according to a Poisson ratio tester. Metallographic treatment is performed on titanium alloy, an etched crystal sample is observed under a metallographic microscope and a transmission electron microscope (TEM), and then a metallographic map and a Fourier transform image are obtained. A dislocation type is determined and a number of dislocation etching pits is measured by combining metallographic map and the Fourier transform image. The dislocation source density (ρₛ) of titanium alloy is calculated through a TEM grid intersection dislocation measurement method. Dislocation obstacle density (ρ_{obs}=2ρₛ, that is, generally twice the dislocation source density) is obtained according to the dislocation source density. Moreover, some other known α-phase crystal parameters are shown in Table 1.

TABLE 1
Some known material parameters in α-phase crystals of titanium alloy

| | | | Dislocation | | | |
|---|---|---|---|---|---|---|
| Dislocation | | Dislocation | source | Glide | | Material |
| segment | Viscosity | obstacle | multiplication | plane | Burgers | related |
| length | coefficient | strength | time | spacing | vector | parameter |
| (Lab/nm) | (Bg/Pa*s) | (τ_{obs}/MPa) | (tnuc/ns) | (d/nm) | (b/nm) | (z, λ) |
| 500 | 1.4 * | 150 | 10 | 100b | 0.295 | 1, 0.5 |
| | 10\hat{ }−4 | | | | | |

The grain refinement analysis region can be understood as a selected region for analyzing a grain refinement degree during titanium alloy ultra-precision cutting. In order to ensure reliability of calculation and reduce a calculation amount, in the embodiment, any fixed region is preferably selected on a titanium alloy workpiece to be machined. Specifically, the step of selecting a grain refinement analysis region on the titanium alloy workpiece to be machined includes:
select the grain refinement analysis region with a preset size on the titanium alloy workpiece to be machined according to an initial cutting position of a cutting tool. The grain refinement analysis region is located just below the initial cutting position. Selection of the preset size can be determined according to the actual situation, which is not specifically limited herein.

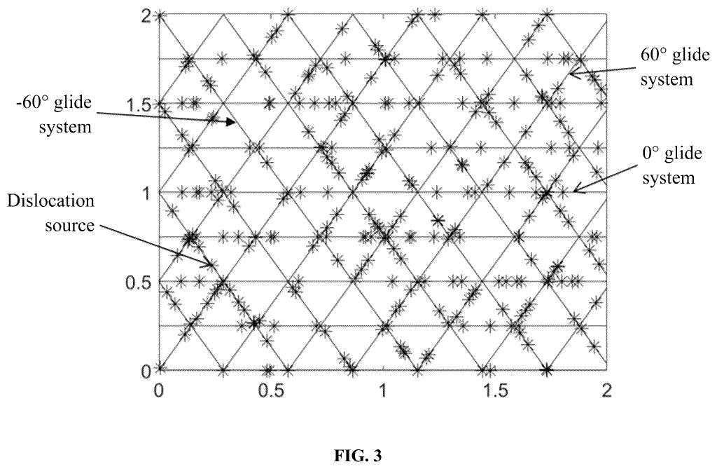

S12, establish a discrete dislocation dynamics model corresponding to the grain refinement analysis region according to the α-phase crystal parameter. The discrete dislocation dynamics model is configured to simulate dislocation behavior in the grain refinement analysis region, and includes simulation calculation processes such as dislocation multiplication, dislocation movement, dislocation annihilation, dislocation pile-up and grain refinement. Specifically, the step of establishing a discrete dislocation dynamics model corresponding to the grain refinement analysis region according to the α-phase crystal parameter includes:
establish an α-phase crystal glide system according to a glide plane spacing and a glide direction in an α phase of titanium alloy, and a relative angle between a glide system and a grain boundary in a unit cell, where a glide plane and a glide direction of a dislocation in the α phase of titanium alloy are {1 0 −1 0} and <1 1 −2 0>, the relative angles between a glide system and a grain boundary in a unit cell is equal to {0°, 60°, −60°}, and glide system directions (dislocation movement direction) of the α-phase crystal glide system include a 0° direction, a 60° direction and a −60° direction;
divide the grain refinement analysis region into a preset number of grain regions with a same size evenly, where the preset number can be arbitrarily selected in principle, but considering that a size of an α-phase grain of titanium alloy is generally in micron level, in the embodiment, it is preferable to evenly divide the grain refinement analysis region with a length u (mm) and a width w (mm) into 100u*100w regions with a same size according to the mode shown in FIG. 2, each region with the same size is a grain, and the size of the grain region is 10 microns;
obtain a number of dislocation sources according to the dislocation source density and a size of the grain refinement analysis region, distribute the dislocation sources in the glide system directions through a normal distribution method evenly, and obtain positions of all the dislocation sources, where the number of dislocation sources nₛ is obtained by multiplying the dislocation source density by the size of the grain refinement analysis region and is expressed as:

$${n}_{s} = u * w * {ρ}_{s} \qquad ( 1 )$$

where u and w denote the length and the width of the grain refinement analysis region respectively, and ρₛ denotes the dislocation source density; and
after the number of dislocation sources in the grain refinement analysis region is obtained by equation (1), a normal distribution method is used to make the first ⅓ dislocation sources located on the glide system in 0° direction, the middle ⅓ dislocation sources located on the glide system in 60° direction, and the last ⅓ dislocation sources located on the glide system in −60° direction, the positions Sₛ of dislocation sources shown in FIG. 3 are obtained, and the positions Sₛ of dislocation sources are unchanged;
arrange one dislocation obstacle in front of and behind each dislocation source in the glide system direction according to the spacing between a dislocation source and an obstacle separately, and obtain positions of all the dislocation obstacles, where the spacing between a dislocation source and an obstacle is expressed as:

$${L}_{obs} = \frac{3}{2 {ρ}_{obs} \cdot d}$$

where L_{obs}, ρ_{obs} and d denote the spacing between a dislocation source and an obstacle, the dislocation obstacle density and the glide plane spacing respectively; correspondingly, the position of a dislocation obstacle can be calculated according to a position of a dislocation source on a same glide system and the corresponding spacing between the dislocation source and the obstacle, and the position S_{obs} of the dislocation obstacle is also fixed; and
it should be noted that after arranged in the grain refinement analysis region, the dislocation sources and the dislocation obstacles remain unchanged in a subsequent analysis prediction process, that is, all dislocation sources are immovable dislocations, a corresponding number of immovable dislocations is equal to a number of dislocation sources, in a subsequent grain refinement analysis process, the number of immovable dislocations nₛ remains unchanged, in an actual cutting process, movable dislocations are continuously generated by the dislocation sources, and a number of movable dislocations n_{dis} is continuously updated, and reference may be made to related description in the grain refinement analysis below for a particular change manner; and
establish the discrete dislocation dynamics model based on discrete dislocation dynamics according to the α-phase crystal glide system, the positions of all the dislocation sources, and the positions of all the dislocation obstacles.

According to the embodiment, dislocation sources are randomly distributed, and dislocation behavior is simulated based on discrete dislocation dynamics. The method is universal, general-purpose and practical, improves the simulation accuracy of dislocation behavior, and further provides reliable guarantee for the accuracy of grain refinement analysis.

S13, perform grain refinement prediction analysis on the grain refinement analysis region according to the discrete dislocation dynamics model in response to ultra-precision cutting of the titanium alloy workpiece to be machined, and obtain a corresponding grain size after cutting.

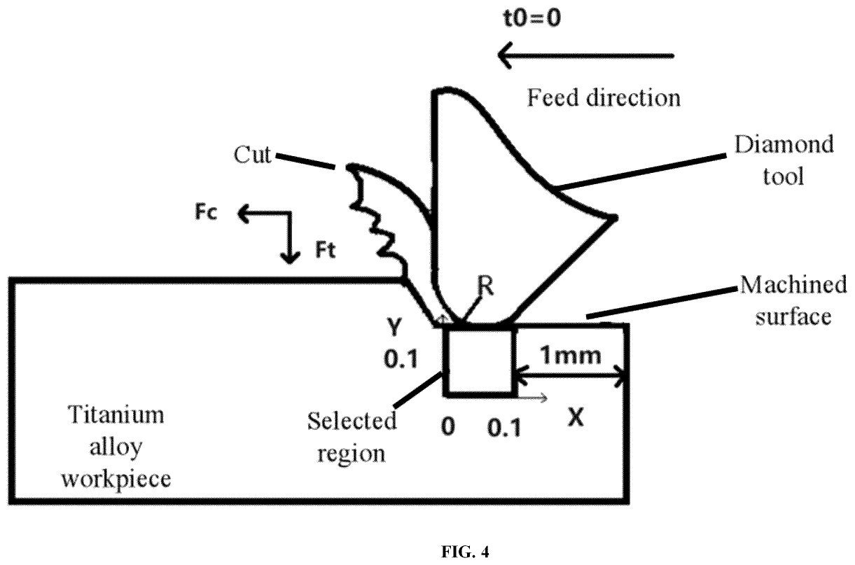

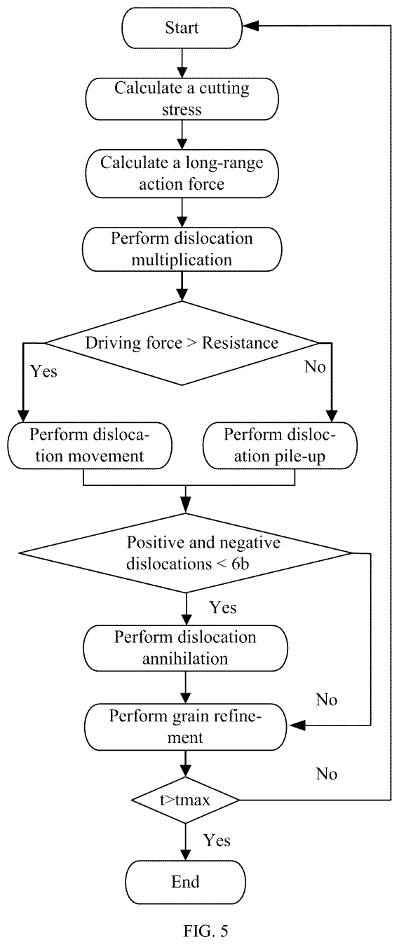

Specifically, the steps of performing grain refinement prediction analysis on the grain refinement analysis region according to the discrete dislocation dynamics model, and obtaining a corresponding grain size after cutting include:
obtain ultra-precision cutting parameters, where the ultra-precision cutting parameters include a corner radius, a cutting speed and a cutting depth;
obtain a calculation prediction time according to the cutting speed and a length of the grain refinement analysis region, take each dislocation source as an immovable dislocation, and initialize a number of dislocations, where the dislocations include movable dislocations and immovable dislocations, and the calculation prediction time can be understood as a time of entire grain refinement analysis during cutting and is expressed as:

$${t}_{max} = 1.5 u / {v}_{t}$$

where tₘₐₓ denotes the calculation prediction time; u denotes a length of the grain refinement analysis region; and νₜ denotes the cutting speed; and
it should be noted that as shown in FIG. 4, the iterative prediction calculation in the embodiment starts from when the cutting tool just cuts to the grain refinement analysis region, at this time, the number of movable dislocations is 0, and an initial value of the number of dislocations is actually the number of dislocation sources; and
perform iterative analysis on grain refinement in the grain refinement analysis region based on the discrete dislocation dynamics model within the calculation prediction time according to the ultra-precision cutting parameters and the α-phase crystal parameters when a cutting tool cuts to the grain refinement analysis region, and obtain the grain size after cutting. An iterative calculation process of the grain size after cutting is shown in FIG. 5, and includes simulation calculation processes of a cutting stress suffered by dislocations, a long-range action force, dislocation multiplication, dislocation movement/dislocation pile-up, dislocation annihilation, grain refinement, etc. in the cutting process. Specifically, the steps of performing iterative analysis on grain refinement in the grain refinement analysis region based on the discrete dislocation dynamics model within the calculation prediction time, and obtaining the grain size after cutting include:
initialize a number of immovable dislocations, a number of movable dislocations and a number of times of grain refinement, where at the beginning of iteration, a moment when the cutting tool just reaches the grain refinement analysis region is recorded as t₀, in this case, the number of immovable dislocations, the number of movable dislocations and the number of times of grain refinement are respectively the number of dislocation sources, 0 and 0; and moreover, a time step of iteration calculation is set as a dislocation multiplication time, it can be understood that the grain refinement prediction analysis is performed once for each dislocation multiplication time;
obtain cutting thrust of the cutting tool at a current moment, and obtain a shear stress and a long-range action resultant stress of each dislocation and each dislocation source according to the cutting thrust and the position of each dislocation, where the step of obtaining a shear stress and a long-range action resultant stress of each dislocation and each dislocation source according to the cutting thrust and the position of each dislocation includes:
obtain the shear stress of each dislocation according to the cutting thrust, where the shear stress is expressed as:

$${τ}_{i} = {σ}_{t} - {ze}^{λ \sqrt{{[ {x}_{i} - ( u - {v}_{t} {t}_{0} ) ]}^{2} + {( {y}_{i} - w )}^{2}}} \qquad ( 2 )$$

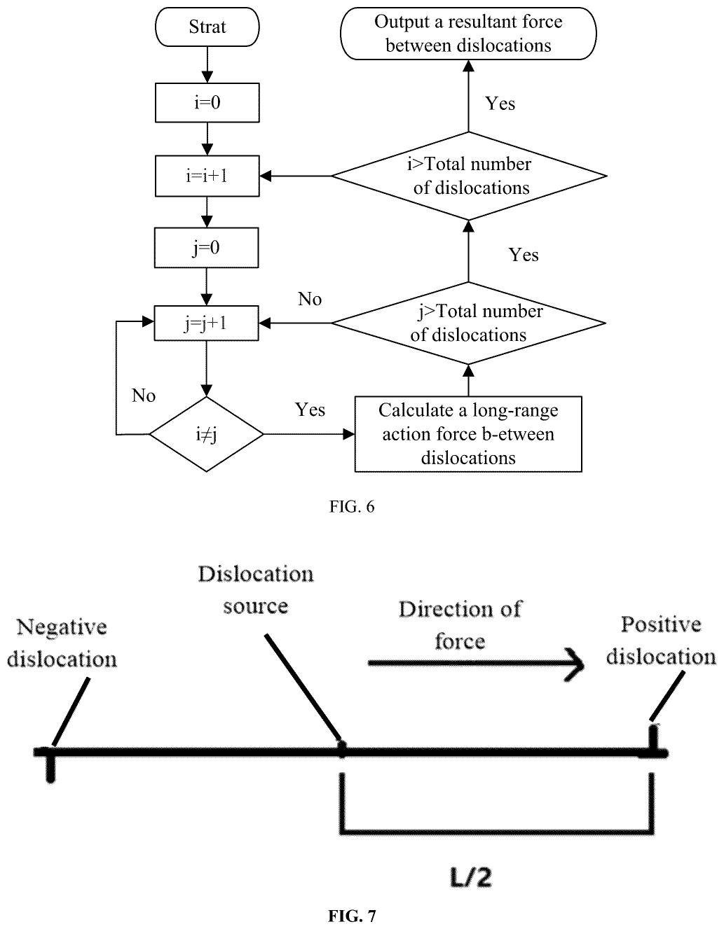

in the equation,

$${σ}_{t} = \frac{{F}_{t}}{W}$$

$$W = {R}^{2} \cdot arccos \frac{R - h}{R} - ( R - h ) \sqrt{{R}^{2} - {( R - h )}^{2}}$$

where τᵢ denotes a shear stress caused by cutting on an ith dislocation; z and λ denote material parameters of titanium alloy; νₜ denotes the cutting speed; (xᵢ, yᵢ) denotes coordinates of the ith dislocation in the grain refinement analysis region; u and w denote a length and a width of the grain refinement analysis region respectively; Fₜ denotes the cutting thrust measured by a dynamometer at moment t₀; σₜ denotes the shear stress of the cutting tool; W denotes a contact area between the cutting tool and the titanium alloy workpiece to be machined; R denotes the corner radius; and h denotes the cutting depth; and
obtain a long-range action force between dislocations according to the position of each dislocation, and obtain the long-range action resultant stress of each dislocation according to the long-range action force between dislocations, where the long-range action resultant stress can be understood as an accumulation of the long-range action forces of all other dislocations on one dislocation, and a specific calculation process is shown in FIG. 6 and expressed as:

$${σ}_{all}^{i} = {\sum }_{j = 1}^{i \neq j} {σ}_{ij} \qquad ( 3 )$$

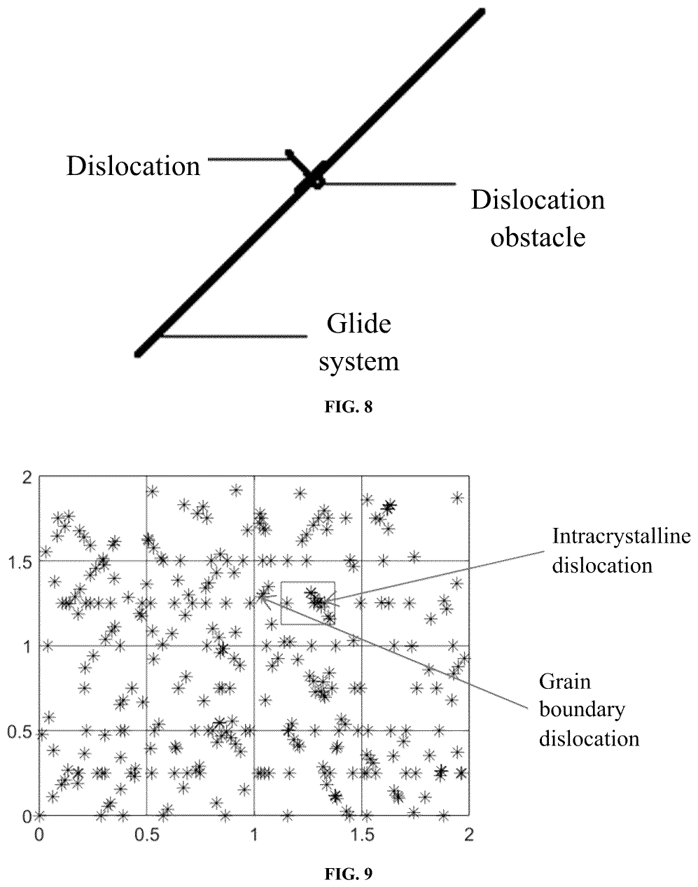

in the equation,

$${σ}_{ij} = \frac{μ b}{2 π ( 1 - v )} \cdot \frac{- 2 {dy}_{ij} \cdot sin 2 θ \cdot {dx}_{ij}^{2} + {dx}_{ij} \cdot cos 2 θ \cdot ( {dx}_{ij}^{2} - {dy}_{ij}^{2} )}{( {dx}_{ij}^{2} + {dy}_{ij}^{2} )} ,$$

$$i , j = 1 , … , {n}_{s} + {n}_{dis} , i \neq j$$

where σₐₗₗⁱ denotes a long-range action resultant stress of the ith dislocation; σᵢⱼ and θ denote a long-range action force and an angle between the ith dislocation and a jth dislocation respectively, and 0°<=θ<180°; dxᵢⱼ and dyᵢⱼ denote distances between the dislocation i and the dislocation j along an x axis and an y axis respectively; and nₛ and n_{dis} denote the number of immovable dislocations and the number of movable dislocations respectively; and it should be noted that considering that different dislocations have different glide system angles, the accuracy of long-range stress calculation can be improved by adding a variable θ to the calculation equation of long-range stress calculation;
calculate an intra-region Peierls-Nabarro stress and a dislocation source multiplication intensity in the grain refinement analysis region according to the shear modulus, the Poisson ratio of titanium alloy, the Burgers vector and the dislocation segment length, where the intra-region Peierls-Nabarro stress is expressed as:

$${σ}_{P - N} = \frac{2 μ}{1 - v} {e}^{\frac{- 4 πξ}{b}} \qquad ( 4 )$$

where σ_{P-N} denotes the intra-region Peierls-Nabarro stress, μ, v, b and ξ denote the shear modulus, the Poisson ratio of titanium alloy, the Burgers vector and a dislocation half-width respectively, and the dislocation half-width has a size of 5b; and
the dislocation source multiplication intensity is expressed as:

$${τ}_{s} = \frac{μ b}{{L}_{ab}} \qquad ( 5 )$$

where τₛ denotes the dislocation source multiplication intensity, and L_{ab} denotes the dislocation segment length;
obtain a corresponding dislocation source resultant stress according to the shear stress and the long-range action resultant stress of each dislocation source, where the dislocation source resultant stress is expressed as:

$${σ}_{source}^{k} = {τ}_{ok} + {σ}_{ok} , \qquad ( 6 )$$

$$k = 1 , … , {n}_{s}$$

where τₒₖ, τₒₖ and τ_{source}^{k} denote a shear stress, a long-range action resultant stress and a dislocation source resultant stress of a kth dislocation source respectively; and it should be noted that when the dislocation source is an immovable dislocation, τₒₖ and τₒₖ are obtained by calculation through equations (2) and (3) respectively, which are not repeated herein;
determine whether the dislocation source resultant stress is great than the dislocation source multiplication intensity, if so, keep a position of the corresponding dislocation source unchanged, generate a pair of positive and negative dislocations on the glide system of the corresponding dislocation source according to a preset distance, and increase the number of movable dislocations, where the preset distance is preferably obtained according to equation (7), and the corresponding multiplied positive and negative dislocations are shown in FIG. 7:

$$L = \frac{μ}{2 π ( 1 - v )} \cdot \frac{b}{{τ}_{s}} \qquad ( 7 )$$

where L denotes a distance between the positive dislocation and the negative dislocation generated by the dislocation source; and
since the positive and negative dislocations generated by the dislocation source can move on the corresponding glide systems, a subsequent running speed and moving positions at different times need to be analyzed through the following method steps, and the behavior of dislocation pile-up and dislocation annihilation that may occur may further be predicted and analyzed, such that the accuracy of grain refinement analysis is guaranteed;
obtain a movement speed of each movable dislocation according to the shear stress, the long-range action resultant stress and the intra-region Peierls-Nabarro stress of each movable dislocation, obtain corresponding obstacle strength according to the movement speed of each movable dislocation and positions of dislocation obstacles of the same glide system, and obtain a current position of the corresponding movable dislocation according to the movement speed and the obstacle strength, where a movement direction of the movable dislocation is parallel to a direction of the glide system where the movable dislocation is located, and the movement speed of the movable dislocation can be obtained by stress calculation, which is expressed as:

$${v}_{{n}_{l}} = \frac{( {τ}_{{n}_{l}} - {σ}_{P - N} + {σ}_{{n}_{l}} ) b}{{B}_{g}} \qquad ( 8 )$$

where νₙ_{ₗ}, τₙ_{ₗ} and σₙ_{ₗ} denote a movement speed, a shear stress and a long-range action resultant stress of a nₗ th movable dislocation respectively; σ_{P-N} denotes the intra-region Peierls-Nabarro stress; and b and B_{g} denote the Burgers vector and the viscosity coefficient respectively;
it should be noted that, considering that in a moving process of a movable dislocation, dislocation pile-up may occur due to an influence of an obstacle strength and a regional Peierls-Nabarro stress received in an actual moving process, resulting in dislocation unable to move normally, that is, a corresponding position does not change; the obstacle strength received in the moving process depends on whether dislocation obstacles are encountered, a specific analysis process is as follows: assuming that the movable dislocation nₗ is subjected to an obstacle strength τ_{obs}^{l}, a corresponding magnitude of the obstacle strength is τ_{obs} only when the movable dislocation passes through the position S_{obs} where the dislocation obstacle is located, that is, when the movable dislocation nₗ appears at the position shown in FIG. 8 when moving, and otherwise, the corresponding magnitude is 0; and specifically, the question whether the movable dislocation nₗ encounters the dislocation obstacle can be determined by the following calculation:
a time t₁ required for the movable dislocation nₗ to reach the dislocation obstacle is calculated according to equation (9), in case that 0<=t₁<t (where t denotes a time step of iterative prediction and is equal to a dislocation multiplication time length), it is determined that the movable dislocation nₗ will encounter the dislocation obstacle in the current motion process, and otherwise, the movable dislocation will not encounter the dislocation obstacle;

$${t}_{1} = ( {S}_{obs} - {S}_{t 0}^{{n}_{l}} ) / {v}_{{n}_{l}} \qquad ( 9 )$$

after the movement speed and the obstacle strength of each movable dislocation are determined through the above method steps, the position of each movable dislocation can be calculated through the following method; and specifically, the step of obtaining a current position of the corresponding movable dislocation according to the movement speed and the obstacle strength includes:
obtain a first movable dislocation position according to the movement speed and an initial position of each movable dislocation, where the first movable dislocation position can be understood as a position of a movable dislocation moved after one dislocation multiplication time under the condition that no dislocation obstacle is encountered normally, and is expressed as:

$${s}_{{t}_{0} + t}^{{n}_{l}} = {s}_{{t}_{0}}^{{n}_{l}} + {v}_{{n}_{l}} t ,$$

$${n}_{l} = 1 , … , {n}_{dis}$$

where νₙ_{ₗ} denotes a movement speed of a nₗ th movable dislocation; sₜ_{₀}ⁿ^{ₗ} and sₜ_{₀}_{⁺¹}ⁿ^{ₗ} denote positions of the nₗth dislocation before and after moving within a first dislocation multiplication time t from moment to respectively;
obtain a corresponding driving force according to the shear stress and the long-range action resultant stress of each movable dislocation, and obtain a corresponding movement resistance according to the intra-region Peierls-Nabarro stress and the corresponding obstacle strength, where the driving force is expressed as:

$${σ}_{driving}^{{n}_{l}} = {τ}_{{n}_{l}} + {σ}_{all}^{{n}_{l}}$$

where σ_{driving}ⁿ^{ₗ}, τₙ_{ₗ} and σₐₗₗⁿ^{ₗ} denote a driving force, a shear stress and a long-range action resultant stress of the nₗ th movable dislocation respectively; and
the movement resistance is expressed as:

$${F}_{obs}^{{n}_{l}} = {τ}_{obs}^{{n}_{l}} + {σ}_{P - N}$$

where F_{obs}ⁿ^{ₗ} and τ_{obs}ⁿ^{ₗ} denote a movement resistance and an obstacle strength of an nₗ th movable dislocation respectively; and
determine whether the driving force of each movable dislocation is greater than the corresponding movement resistance, if so, take the first movable dislocation position as the current position of a movable dislocation, and otherwise, determine that dislocation pile-up occurs in the corresponding movable dislocation, and take the corresponding initial position as the current position of a movable dislocation, where
it should be noted that, in principle, after the above dislocation pile-up determination, accurate positions of all movable dislocations after one dislocation multiplication time shown in equation (10) can be obtained, however, in an actual dislocation movement process, a situation where a spacing between a positive dislocation and a negative dislocation on the same glide system is too small, and dislocation annihilation occurs, that is, two movable dislocations disappear at the same time may also occur; and in view of that, in order to ensure the comprehensiveness and accuracy of dislocation behavior simulation, the embodiment further identifies possible dislocation annihilation behavior, to update the number of effective movable dislocations in real time and ensure the accuracy of subsequent iterative analysis;

$${s}_{{t}_{0} + t}^{{n}_{l}} = \{ \begin{matrix}{s}_{{t}_{0}}^{{n}_{l}} + {v}_{{n}_{l}} t , No dislocation pile - up occurs \\ {s}_{{t}_{0}}^{{n}_{l}} , Disocation pile - up occurs\end{matrix} \qquad ( 10 )$$

obtain a corresponding movable dislocation spacing according to the current position of each movable dislocation, determine whether dislocation annihilation occurs according to the movable dislocation spacing, remove the corresponding movable dislocation when the dislocation annihilation occurs, and reduce the number of movable dislocation, where the movable dislocation spacing can be understood as a distance between any pair of movable dislocations and is expressed as:

$${L}_{s}^{lm} = \vert {S}_{t 0 + t}^{{n}_{l}} - {S}_{t 0 + t}^{{n}_{m}} \vert$$

where Lₛ^{lm} denotes a movable dislocation spacing between a nₗ th movable dislocation and a nₘ th movable dislocation; and Sₜ₀₊ₜⁿ^{ₗ} and Sₜ₀₊ₜⁿ^{ₗ} denote current positions of movable dislocations corresponding to the nₗth movable dislocation and the nₘ th movable dislocation respectively at moment t0+t;
count a number of grain boundary dislocations in each grain region according to the current positions of all movable dislocations and the positions of the dislocation sources, and obtain a corresponding grain boundary torsion angle according to the number of grain boundary dislocations in each grain region, where the number of grain boundary dislocations can be understood as the number of dislocations located in the grain region but not in a crystal center region with a size of ( 1/200)*( 1/200), and as shown in FIG. 9, dislocations located in the crystal center region are intracrystalline dislocations, and dislocations located in the grain boundary region are grain boundary dislocations; correspondingly, a grain boundary torsion angle of each grain is expressed as:

$$\\ \begin{matrix}{θ}_{b}^{{}_{} q} = arc tan ( b \sqrt{\frac{{n}_{bound}^{{}_{} q}}{{A}_{bound}^{{}_{} q}}} ) & ( 11 )\end{matrix}$$

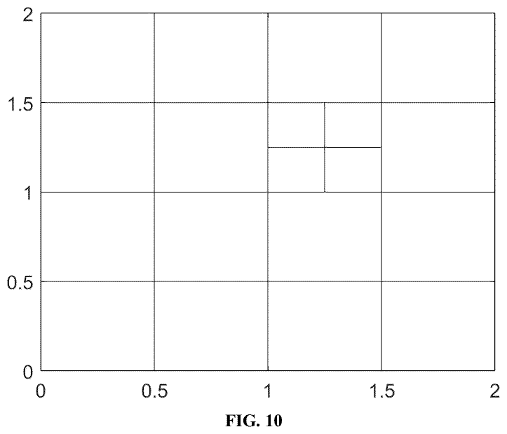

in the equation,

$$\\ {A}_{bound}^{{}_{} q} = ( 1 - \frac{1}{4} ) * {D}^{{}_{} 2}$$

where θ_{b}^{q}, A_{bound}^{q} and n_{bound}^{q} denote a grain boundary torsion angle, a grain boundary region area and a number of grain boundary dislocations of a qth grain region respectively; D denotes a grain size length; and b denotes the Burgers vector;
determine whether a grain refinement region exists according to the grain boundary torsion angle, divide the grain refinement region into four grain sub-regions with a same size, and increase the number of times of grain refinement, where a specific process of determining whether a grain refinement region exists according to the grain boundary torsion angle can be understood as determining whether the grain boundary torsion angle of each grain is greater than a preset degree (for example, 5°), if so, that is, the grain boundary torsion angle of a certain grain is greater than the preset degree, it is determined that grain refinement occurs in the grain, and otherwise, no grain refinement occurs; moreover, grain refinement can be understood as a process of dividing a grain into four sub-grains with a same size, that is, as shown in FIG. 10, when one grain is refined, the number of corresponding grains increases by 3, and the corresponding number of times n of grain refinements increases by 1; and accordingly, it can be considered that one iteration prediction of grain refinement is completed, then relevant iteration calculation parameters need to be updated from obtaining the cutting thrust of the cutting tool, and a new grain refinement iteration prediction is executed until a preset calculation prediction time is reached; and
update a current analysis moment according to the dislocation multiplication time, obtain current cutting thrust of the cutting tool, update the cutting thrust according to the current cutting thrust, continue a next round of grain refinement analysis, stop iteration until a preset calculation prediction time is reached, and obtain the grain size after cutting according to the number of times of grain refinement. The grain size after cutting is expressed as:

$$\\ \begin{matrix}{D}_{new} = \sqrt{\frac{u * w}{100 u * 100 w + 3 * n}} & ( 12 )\end{matrix}$$

where D_{new} denotes the grain size after cutting; n denotes the number of times of grain refinement; and u and w denote a length and a width of the grain refinement analysis region respectively.

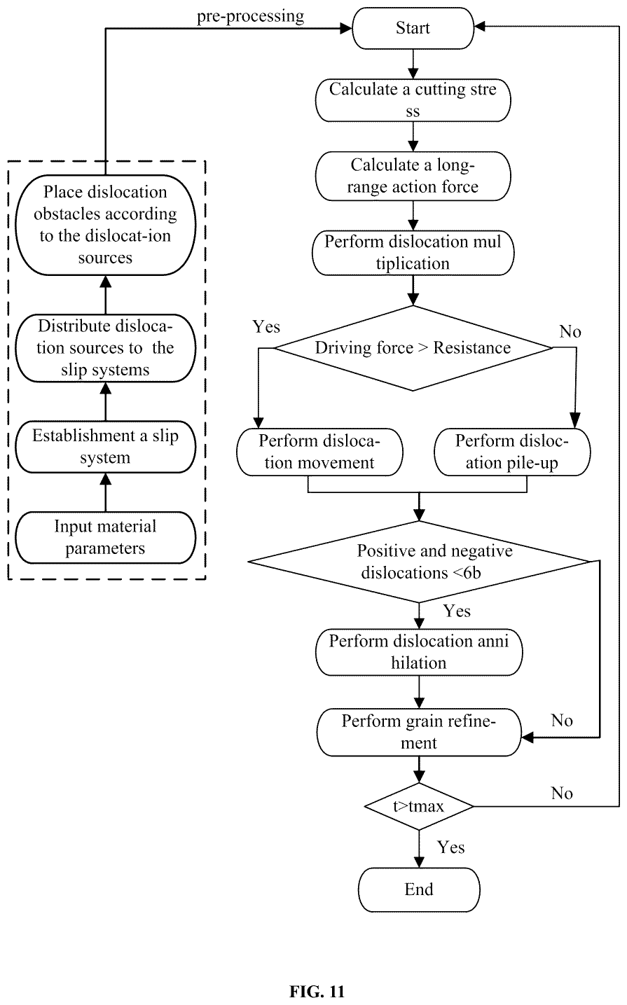

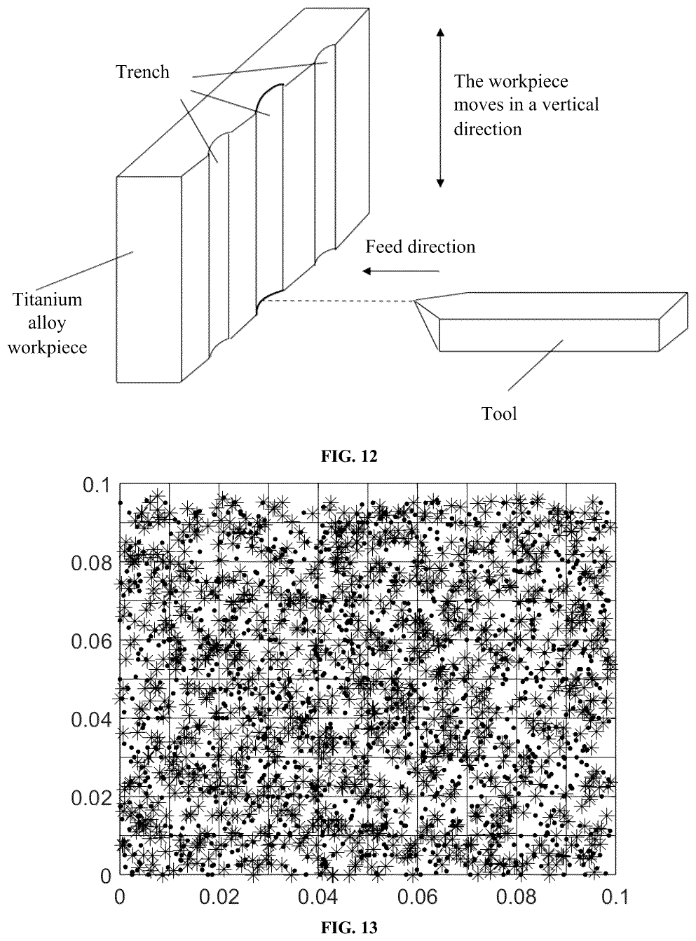

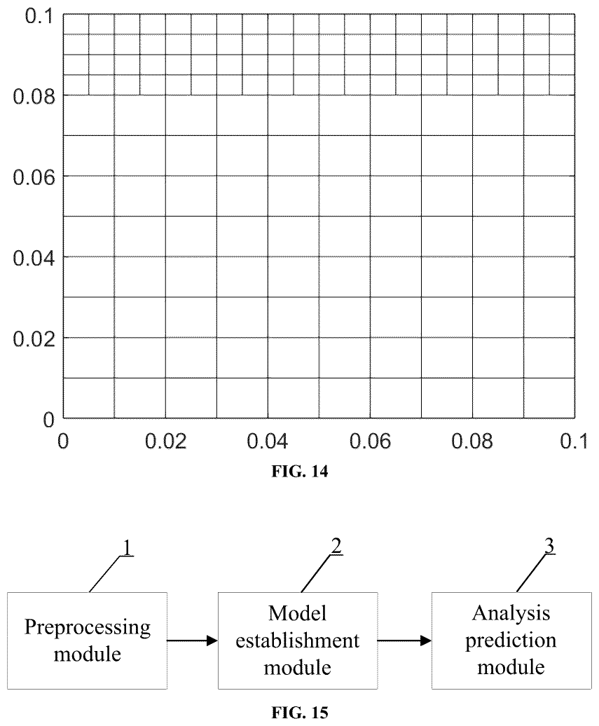

Furthermore, in order to verify the effectiveness of the method for predicting grain refinement of a machined surface of titanium alloy subjected to ultra-precision cutting provided in the present disclosure, the embodiment also performs grain refinement simulation prediction on cutting of a titanium alloy sample (related α-phase crystal parameters: b=0.295 nm, μ=113.76 GPa, ν=0.342, and ρₛ=1.2*10¹³ m⁻²; a size of a grain refinement analysis region 1 mm away from a cutting position of a cutting tool: μ=0.1 mm, w=0.1 mm; and cutting depth h=30 um, and cutting speed vₜ=5 m/s) shown in FIG. 12 according to a detailed flow shown in FIG. 11. A dislocation behavior simulation result shown in FIG. 13 is obtained (‘*’ denotes a dislocation source, and ‘.’ denotes a movable dislocation). According to the dislocation distribution shown in FIG. 13, the grain boundary torsion angle of each crystal grain is calculated, it is determined whether grain refinement occurs in each crystal grain, an increase amount of times of grain refinement in this iteration is obtained, and the next iteration is performed. By completing all iterative calculations, the number of times of grain refinement n=20 can finally be obtained. As shown in FIG. 14, accordingly, an average grain size in the selected region after micro-cutting is calculated to be equal to 7.9057 microns, which is obviously reduced compared with an original grain size of 10 microns. Therefore, a subsequent machining process needs to be designed according to the change of the grain size. For example, in case that the change of the average grain size is not large, the grain refinement has little influence, material parameters such as an elastic modulus of titanium alloy have small variation, and the grain refinement does not need to be considered in the subsequent machining process. In case that the grain size changes greatly, the material parameters such as an elastic modulus of titanium alloy change greatly, and the subsequent machining process needs to be modified according to grain refinement. Moreover, in case that the number of times of grain refinement n is too large, some material parameters of titanium alloy change obviously, it is difficult to design the subsequent machining process, and then a cutting depth can be modified to reduce grain refinement, such that the change of the grain size is reduced.

In the embodiment of the present application, by using randomly distributed dislocation sources (instead of continuous ones) and combining the dislocation behavior in the ultra-precision cutting simulated based on discrete dislocation dynamics, the microstructure change of grain refinement of titanium alloy is deduced, and iterative calculation analysis is carried out on the grain refinement of the machined surface in the α phase of the titanium alloy, such that efficient and accurate prediction of the grain size of the machined surface after cutting is implemented. Not only operation is simple, and calculation amount is also reduced. Compared with a traditional finite element simulation method, the method in the present application is improved in universality and feasibility, and can directly, quickly and accurately observe and evaluate quality of a machined surface, provides reliable guarantee for effective designation of subsequent machining process design, and has high application value.

It should be noted that while various steps in the above flowcharts are shown in sequence as indicated by arrows, the steps are not necessarily performed in sequence in the order indicated by the arrows. The execution of these steps is not strictly limited in order and can be executed in other orders, unless explicitly stated herein.

In an embodiment, as shown in FIG. 15, a system for predicting grain refinement of a machined surface of titanium alloy subjected to ultra-precision is provided. The system includes:
a preprocessing module 1 configured to obtain an α-phase crystal parameter of a titanium alloy workpiece to be machined in advance, and select a grain refinement analysis region on the titanium alloy workpiece to be machined;
a model establishment module 2 configured to establish a discrete dislocation dynamics model corresponding to the grain refinement analysis region according to the α-phase crystal parameter, where the discrete dislocation dynamics model is configured to simulate dislocation behavior in the grain refinement analysis region; and
an analysis prediction module 3 configured to perform grain refinement prediction analysis on the grain refinement analysis region according to the discrete dislocation dynamics model in response to ultra-precision cutting of the titanium alloy workpiece to be machined, and obtain a corresponding grain size after cutting.

For the specific definition of the system for predicting grain refinement of a machined surface of titanium alloy subjected to ultra-precision, reference may be made to the definition of the method for predicting grain refinement of a machined surface of titanium alloy subjected to ultra-precision above, and corresponding technical effects can also be obtained equivalently, which is not repeated herein. Each module in the system for predicting grain refinement of a machined surface of titanium alloy subjected to ultra-precision can be fully or partially achieved by software, hardware and their combinations. Each module may be embedded in or independent of a processor of a computer apparatus in a hardware form, or may be stored in a memory of the computer apparatus in a software form, such that the processor can call the modules and execute operations corresponding to the above modules.

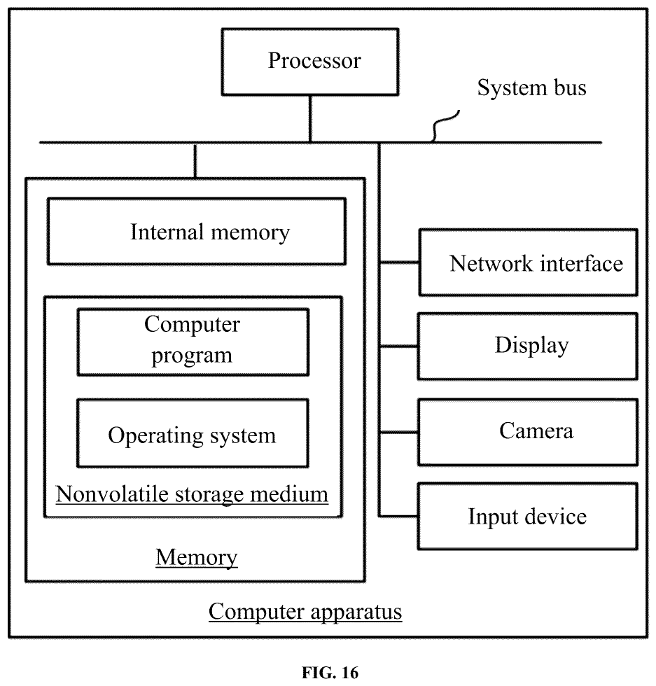

FIG. 16 is an internal structure diagram of a computer apparatus according to an embodiment. The computer apparatus may be a terminal or a server. As shown in FIG. 16, the computer apparatus includes a processor, a memory, a network interface, a display, and an input device connected by a system bus. The processor of the computer apparatus is configured to provide calculation and control capabilities. The memory of the computer apparatus includes a nonvolatile storage medium, and an internal memory. The nonvolatile storage medium stores an operating system and a computer program. The internal memory provides an environment for execution of the operating system and computer programs in the nonvolatile storage medium. The network interface of the computer apparatus is configured to communicate with an external terminal through a network connection. The computer program implements a method for predicting grain refinement of a machined surface of titanium alloy subjected to ultra-precision cutting when executed by the processor The display screen of the computer apparatus may be a liquid crystal display screen or an electronic ink display screen. The input device of the computer apparatus may be a touch layer covering the display screen, a key, a trackball or a touch pad arranged on a housing of the computer apparatus, or an external keyboard, a touch pad or a mouse.

Those skilled in the art will appreciate that the structure shown in FIG. 16 is merely a block diagram of a portion of the structure related to the solution of the present application, and does not constitute a limitation on the computer apparatus to which the solution of the present application is applied. A specific computing apparatus may include more or less components than shown in the figure, or combine some components, or have the same component arrangement.

In an embodiment, a computer apparatus is provided. The computer apparatus includes a memory, a processor, and a computer program stored on the memory and runnable on the processor, where the processor implements steps of the above method when executing the computer program.

In an embodiment, a computer-readable storage medium is provided. The computer-readable storage medium stores a computer program, where the computer program implements steps of the above method when executed by a processor.

In summary, the embodiments of the present disclosure provides a method and system for predicting grain refinement of a machined surface of titanium alloy subjected to ultra-precision cutting, a computer apparatus and a storage medium. The method for predicting grain refinement of a machined surface of titanium alloy subjected to ultra-precision cutting achieves a technical solution of obtaining an α-phase crystal parameter of a titanium alloy workpiece to be machined in advance, selecting a grain refinement analysis region on the titanium alloy workpiece to be machined, establishing a discrete dislocation dynamics model corresponding to the grain refinement analysis region according to the α-phase crystal parameter, performing grain refinement prediction analysis on the grain refinement analysis region according to the discrete dislocation dynamics model when the titanium alloy workpiece to be machined is subjected to ultra-precision cutting, and obtaining a corresponding grain size after cutting. According to method, the dislocation behavior inside titanium alloy during machining is calculated and simulated based on discrete dislocation dynamics, a microstructure change of titanium alloy grain refinement is deduced, and a grain size of the machined surface after cutting is predicted, such that quality of the machined surface can be evaluated intuitively, rapidly and accurately, reliable guarantee for optimizing a subsequent machining process is provided, and the method has high application value.

Each embodiment in this specification is described in a progressive manner, the same or similar parts of the embodiments can be referred to each other directly, and differences between each embodiment and other embodiments are emphasized. Particularly, since the system embodiments are similar to the method embodiments basically, the description is simpler, and reference can be made to the description of the method embodiments. It should be noted that the various technical features of the embodiments described above may be arbitrarily combined, and not all possible combinations of the various technical features in the embodiments described above are described for brevity of description. However, to the extent that there is no contradiction in the combination of the technical features, the combination should be considered to fall within the scope of the specification.

The embodiments described above express only some preferred implementations of the present application, which are specific and detailed in description, but cannot be construed as limiting the scope of the invention patent accordingly. It should be noted that a plurality of improvements and substitutions may also be made by those of ordinary skill in the art without departing from the technical principles of the present disclosure, which should also be considered as the scope of protection of the present application. Therefore, the scope of protection of the present application patent shall be subject to the scope of protection of the claims.

What is claimed is:

1. A method for predicting grain refinement of a machined surface of titanium alloy subjected to ultra-precision cutting, comprising:
obtaining an α-phase crystal parameter of a titanium alloy workpiece to be machined in advance, and selecting a grain refinement analysis region on the titanium alloy workpiece to be machined;
establishing a discrete dislocation dynamics model corresponding to the grain refinement analysis region according to the α-phase crystal parameter, wherein the discrete dislocation dynamics model is configured to simulate dislocation behavior in the grain refinement analysis region; and
performing grain refinement prediction analysis on the grain refinement analysis region according to the discrete dislocation dynamics model in response to ultra-precision cutting of the titanium alloy workpiece to be machined, and obtaining a corresponding grain size after cutting.

2. The method for predicting grain refinement of a machined surface of titanium alloy subjected to ultra-precision cutting according to claim 1, wherein the step of selecting a grain refinement analysis region on the titanium alloy workpiece to be machined comprises:
selecting the grain refinement analysis region with a preset size on the titanium alloy workpiece to be machined according to an initial cutting position of a cutting tool, wherein the grain refinement analysis region is located just below the initial cutting position.

3. The method for predicting grain refinement of a machined surface of titanium alloy subjected to ultra-precision cutting according to claim 1, wherein the α-phase crystal parameters comprise dislocation source density, a spacing between a dislocation source and an obstacle, and a glide plane spacing; and
the step of establishing a discrete dislocation dynamics model corresponding to the grain refinement analysis region according to the α-phase crystal parameter comprises:
establishing an α-phase crystal glide system according to a glide plane spacing and a glide direction in an α phase of titanium alloy, and a relative angle between a glide system and a grain boundary in a unit cell, wherein glide system directions of the α-phase crystal glide system comprise a 0° direction, a 60° direction and a −60° direction;
dividing the grain refinement analysis region into a preset number of grain regions with a same size evenly;
obtaining a number of dislocation sources according to the dislocation source density and a size of the grain refinement analysis region, distributing the dislocation sources in the glide system directions through a normal distribution method evenly, and obtaining positions of all the dislocation sources;
arranging one dislocation obstacle in front of and behind each dislocation source in the glide system direction according to the spacing between a dislocation source and an obstacle separately, and obtaining positions of all the dislocation obstacles; and
establishing the discrete dislocation dynamics model based on discrete dislocation dynamics according to the α-phase crystal glide system, the positions of all the dislocation sources, and the positions of all the dislocation obstacles.

4. The method for predicting grain refinement of a machined surface of titanium alloy subjected to ultra-precision cutting according to claim 3, wherein the steps of performing grain refinement prediction analysis on the grain refinement analysis region according to the discrete dislocation dynamics model, and obtaining a corresponding grain size after cutting comprise:
obtaining ultra-precision cutting parameters, wherein the ultra-precision cutting parameters comprise a corner radius, a cutting speed and a cutting depth;
obtaining a calculation prediction time according to the cutting speed and a length of the grain refinement analysis region, taking each dislocation source as an immovable dislocation, and initializing a number of dislocations, wherein the dislocations comprise movable dislocations and immovable dislocations; and
performing iterative analysis on grain refinement in the grain refinement analysis region based on the discrete dislocation dynamics model within the calculation prediction time according to the ultra-precision cutting parameters and the α-phase crystal parameters when a cutting tool cuts to the grain refinement analysis region, and obtaining the grain size after cutting.

5. The method for predicting grain refinement of a machined surface of titanium alloy subjected to ultra-precision cutting according to claim 4, wherein the α-phase crystal parameters further comprise a shear modulus, a Poisson ratio of titanium alloy, a Burgers vector, a dislocation segment length, a dislocation multiplication time, a viscosity coefficient and dislocation obstacle strength; and
the steps of performing iterative analysis on grain refinement in the grain refinement analysis region based on the discrete dislocation dynamics model within the calculation prediction time, and obtaining the grain size after cutting comprise:
initializing a number of immovable dislocations, a number of movable dislocations and a number of times of grain refinement;
obtaining cutting thrust of the cutting tool at a current moment, and obtaining a shear stress and a long-range action resultant stress of each dislocation and each dislocation source according to the cutting thrust and the position of each dislocation;
calculating an intra-region Peierls-Nabarro stress and a dislocation source multiplication intensity in the grain refinement analysis region according to the shear modulus, the Poisson ratio of titanium alloy, the Burgers vector and the dislocation segment length, wherein the intra-region Peierls-Nabarro stress is expressed as:
$$\\ {σ}_{P - N} = \frac{2 μ}{1 - v} {e}^{{}_{} \frac{- 4 π ξ}{b}}$$
wherein σ_{P-N} denotes the intra-region Peierls-Nabarro stress, and μ, v, b and ξ denote the shear modulus, the Poisson ratio of titanium alloy, the Burgers vector and a dislocation half-width respectively; and
the dislocation source multiplication intensity is expressed as:
$$\\ {τ}_{s} = \frac{μ b}{{L}_{ab}}$$
wherein τₛ denotes the dislocation source multiplication intensity, and L_{ab} denotes the dislocation segment length;
obtaining a corresponding dislocation source resultant stress according to the shear stress and the long-range action resultant stress of each dislocation source, wherein the dislocation source resultant stress is expressed as:
$$\\ {σ}_{source}^{{}_{} k} = {τ}_{ok} + {σ}_{ok} , k = 1 , … , {n}_{s}$$
wherein τₒₖ, σₒₖ and σ_{source}^{k} denote a shear stress, a long-range action resultant stress and a dislocation source resultant stress of a kth dislocation source respectively;
determining whether the dislocation source resultant stress is great than the dislocation source multiplication intensity, if so, keeping a position of the corresponding dislocation source unchanged, generating a pair of positive and negative dislocations on the glide system of the corresponding dislocation source according to a preset distance, and increasing the number of movable dislocations;
obtaining a movement speed of each movable dislocation according to the shear stress, the long-range action resultant stress and the intra-region Peierls-Nabarro stress of each movable dislocation, obtaining corresponding obstacle strength according to the movement speed of each movable dislocation and positions of dislocation obstacles of the same glide system, and obtaining a current position of the corresponding movable dislocation according to the movement speed and the obstacle strength;
obtaining a corresponding movable dislocation spacing according to the current position of each movable dislocation, determining whether dislocation annihilation occurs according to the movable dislocation spacing, removing the corresponding movable dislocation when the dislocation annihilation occurs, and reducing the number of movable dislocation;
counting a number of grain boundary dislocations in each grain region according to the current positions of all movable dislocations and the positions of the dislocation sources, and obtaining a corresponding grain boundary torsion angle according to the number of grain boundary dislocations in each grain region;
determining whether a grain refinement region exists according to the grain boundary torsion angle, dividing the grain refinement region into four grain sub-regions with a same size, and increasing the number of times of grain refinement; and
updating a current analysis moment according to the dislocation multiplication time, obtaining current cutting thrust of the cutting tool, updating the cutting thrust according to the current cutting thrust, continuing a next round of grain refinement analysis, stopping iteration until a preset calculation prediction time is reached, and obtaining the grain size after cutting according to the number of times of grain refinement.

6. The method for predicting grain refinement of a machined surface of titanium alloy subjected to ultra-precision cutting according to claim 5, wherein the step of obtaining a shear stress and a long-range action resultant stress of each dislocation and each dislocation source according to the cutting thrust and the position of each dislocation comprises:
obtaining the shear stress of each dislocation according to the cutting thrust, wherein the shear stress is expressed as:
$$\\ {τ}_{i} = {σ}_{t} - 𝓏 {e}^{{}_{} λ \sqrt{{[ {x}_{i} - ( u - {v}_{t} {t}_{0} ) ]}^{2} + {( {y}_{i} - w )}^{2}}}$$
in the equation,
$$\\ {σ}_{t} = \frac{{F}_{t}}{S}$$
$$\\ S = {R}^{{}_{} 2} \cdot arc cos \frac{R - h}{R} - ( R - h ) \sqrt{{R}^{{}_{} 2} - {( R - h )}^{2}}$$
wherein τᵢ denotes a shear stress caused by cutting on an ith dislocation; z and λ denote material parameters of titanium alloy; vₜ denotes the cutting speed; (xᵢ, yᵢ) denotes coordinates of the ith dislocation in the grain refinement analysis region; u and w denote a length and a width of the grain refinement analysis region respectively; Fₜ denotes the cutting thrust; σₜ denotes the shear stress of the cutting tool; S denotes a contact area between the cutting tool and the titanium alloy workpiece to be machined; R denotes the corner radius; and h denotes the cutting depth; and
obtaining a long-range action force between dislocations according to the position of each dislocation, and obtaining the long-range action resultant stress of each dislocation according to the long-range action force between dislocations, wherein the long-range action resultant stress is expressed as:
$$\\ {σ}_{all}^{{}_{} i} = {\sum }_{j = 1}^{i \neq j} {σ}_{ij}$$
in the equation,
$$\\ {σ}_{ij} = \frac{μ b}{2 π ( 1 - v )} \cdot \frac{- 2 {dy}_{ij} \cdot sin 2 θ \cdot {dx}_{ij}^{{}_{} 2} + {dx}_{ij} \cdot cos 2 θ \cdot ( {dx}_{ij}^{{}_{} 2} - {dy}_{ij}^{{}_{} 2} )}{( {dx}_{ij}^{{}_{} 2} + {dy}_{ij}^{{}_{} 2} )} ,$$
$$\\ i , j = 1 , … , {n}_{s} + {n}_{dis} , i \neq j$$
wherein σₐₗₗⁱ denotes a long-range action resultant stress of the ith dislocation; σᵢⱼ and θ denote a long-range action force and an angle between the ith dislocation and a jth dislocation respectively; dxᵢⱼ and dyᵢⱼ denote distances between the dislocation i and the dislocation j along an x axis and an y axis respectively; and nₛ and n_{dis} denote the number of immovable dislocations and the number of movable dislocations respectively.

7. The method for predicting grain refinement of a machined surface of titanium alloy subjected to ultra-precision cutting according to claim 5, wherein the step of obtaining a current position of the corresponding movable dislocation according to the movement speed and the obstacle strength comprises:
obtaining a first movable dislocation position according to the movement speed and an initial position of each movable dislocation, wherein the first movable dislocation position is expressed as:
$$\\ {s}_{{t}_{0} + t}^{{}_{} {n}_{l}} = {s}_{{t}_{0}}^{{}_{} {n}_{l}} + {v}_{{n}_{l}} t , {n}_{l} = 1 , … , {n}_{dis}$$
in the equation,
$$\\ {v}_{{n}_{l}} = \frac{( {τ}_{{n}_{l}} - {σ}_{P - N} + {σ}_{{n}_{l}} ) b}{{B}_{g}}$$
wherein νₙ_{ᵢ}, τₙ_{ₗ} and τₙ_{ₗ} denote a movement speed, a shear stress and a long-range action resultant stress of a nₗ th movable dislocation respectively; σ_{P-N} denotes the intra-region Peierls-Nabarro stress; b and B_{g} denote the Burgers vector and the viscosity coefficient respectively; sₜ_{₀}ⁿ^{ₗ} and sₜ_{₀₊ₜ}ⁿ^{ₗ} denote positions of the nₗ th dislocation before and after moving within a first dislocation multiplication time t from moment to respectively;
obtaining a corresponding driving force according to the shear stress and the long-range action resultant stress of each movable dislocation, and obtaining a corresponding movement resistance according to the intra-region Peierls-Nabarro stress and the corresponding obstacle strength; and
determining whether the driving force of each movable dislocation is greater than the corresponding movement resistance, if so, taking the first movable dislocation position as the current position of a movable dislocation, and otherwise, determining that dislocation pile-up occurs in the corresponding movable dislocation, and taking the corresponding initial position as the current position of a movable dislocation.

8. The method for predicting grain refinement of a machined surface of titanium alloy subjected to ultra-precision cutting according to claim 5, wherein the grain boundary torsion angle is expressed as:
$$\\ {θ}_{b}^{{}_{} q} = arc tan ( b \sqrt{\frac{{n}_{bound}^{{}_{} q}}{{A}_{bound}^{{}_{} q}}} )$$
in the equation,
$$\\ {A}_{bound}^{{}_{} q} = ( 1 - \frac{1}{4} ) * {D}^{{}_{} 2}$$
wherein σ_{b}^{q}, A_{bound}^{q} and bound n_{bound}^{q} denote a grain boundary torsion angle, a grain boundary, region area and a number of grain boundary dislocations of a qth grain region respectively; D denotes a grain size length; and b denotes the Burgers vector.

9. The method for predicting grain refinement of a machined surface of titanium alloy subjected to ultra-precision cutting according to claim 5, wherein the grain size after cutting is expressed as:
$$\\ {D}_{new} = \sqrt{\frac{u * w}{100 u * 100 w + 3 * n}}$$
wherein D_{new} denotes the grain size after cutting; n denotes the number of times of grain refinement; and u and w denote a length and a width of the grain refinement analysis region respectively.

10. A system for predicting grain refinement of a machined surface of titanium alloy subjected to ultra-precision cutting, comprising:
a preprocessing module configured to obtain an α-phase crystal parameter of a titanium alloy workpiece to be machined in advance, and select a grain refinement analysis region on the titanium alloy workpiece to be machined;
a model establishment module configured to establish a discrete dislocation dynamics model corresponding to the grain refinement analysis region according to the α-phase crystal parameter, wherein the discrete dislocation dynamics model is configured to simulate dislocation behavior in the grain refinement analysis region; and
an analysis prediction module configured to perform grain refinement prediction analysis on the grain refinement analysis region according to the discrete dislocation dynamics model in response to ultra-precision cutting of the titanium alloy workpiece to be machined, and obtain a corresponding grain size after cutting.
